(19) United States
(12) Patent Application Publication
(10) Pub. No.: US 2018/0147357 A1
(43) Pub. Date: May 31, 2018

(54) DELIVERY METHOD FOR PREPARATION AND ADMINISTRATION OF PHARMACEUTICAL COMPOSITIONS

(71) Applicants: Amir Marashi, Charlotte, NC (US); Jack Krajekian, Charlotte, NC (US)

(72) Inventors: Amir Marashi, Charlotte, NC (US); Jack Krajekian, Charlotte, NC (US)

(21) Appl. No.: 15/363,887

(22) Filed: Nov. 29, 2016

(51) Int. Cl. A61M 5/28; A61M 5/24; A61J 1/20

(52) U.S. Cl. CPC A61M 5/285; A61M 5/24; A61M 2205/582; A61M 2205/581; A61J 1/2093

(57) ABSTRACT

Carpule systems and methods of making and using the same are disclosed. In one embodiment, a carpule system includes a carpule having a carpule body that defines a first chamber and a second chamber. A divider can be disposed between the first and second chambers for isolating a first material in the first chamber from a second material in the second chamber. The carpule system may also include a carpule mixing device that displaces a portion of the divider for mixing the first and second materials to form a mixture and a carpule syringe that dispenses an amount of the mixture. The amount of the mixture being dispensed is measured via an audible sound and/or a tactile sensation provided by the carpule syringe. Systems and methods herein advantageously obviate the need for mixing powdered material via multiple syringes, reduce waste and cost, and improve the accuracy of administering pharmaceutical compositions.

### FIELD

This application relates to carpules and, more particularly, to carpule systems and methods of making and using carpule systems, including for making and administering a composition to a patient.

### BACKGROUND

Syringes are commonly used to deliver medicinal drugs including pharmaceutical compositions, fluids, and/or various types of solutions to patients, for example, in the medical and dental fields. In cases involving powdered material(s) (e.g., powdered medicine), multiple syringes can be used to prepare and deliver a medicinal drug to a patient. For example, a first syringe is used to introduce a liquid solvent to the powdered material and then a second syringe is used to inject the resultant solution into the patient. Unfortunately, using multiple syringes to mix and deliver a solution formed from powdered material to a patient contributes to waste, formation of residual medication, contamination, introduction of air inside the syringe and/or the solution, and/or human error associated with mixing an incorrect ratio of liquid to powder.

In other instances, syringes can be used to dispense and administer the contents of a carpule to a patient. For example, a carpule can be prefilled with a medicinal drug and then inserted into a carpule syringe. The carpule syringe can then dispense the medicinal drug from the carpule into the patient. However, previous systems using carpule syringes can be problematic or unworkable for cases involving powdered materials, since waste, contamination, and/or inaccurate dosing can occur, for least the same reasons identified above for the use of multiple syringes.

Thus, a need exists for improved carpule systems and methods of making and using the same, for example, that improve the preparation and administration of pharmaceutical compositions formed from powdered material.

### SUMMARY

Carpule devices, systems, and methods of making and using the same are described herein. Such systems and methods are advantageous compared to existing systems and methods. For example, in some instances, carpule systems and methods described herein advantageously introduce and mix the contents of a multi-chamber carpule to form one or more doses or units of a solution, which have an extremely accurate and precise concentration. Carpule systems and methods described herein obviate the need to use multiple syringes to prepare, mix, and dispense a solution and minimize introduction of air or other contaminants into the solution and/or materials forming the solution. Carpule systems described herein are cost efficient by virtue of minimizing waste and human error associated with calculating, measuring, and/or preparing mixtures. Carpule systems described herein can also mix, dispense, and administer a volume of a medicinal drug to a patient, whereby the dose or unit of the drug being dispensed and administered to the patient is easily measurable via an audible sound and/or a tactile sensation provided by a carpule syringe. Additionally, systems and methods described herein can dispense or administer a volume of a liquid composition to one or more sites or locations (e.g., within a patient) without the loss of one or more individual droplets of the liquid composition following the desired dispensing or administering. Moreover, systems and methods described herein can achieve this result without the need to “pull back” on a syringe plunger following the desired dispensing or administering, including in between dispensing sites or locations. Further, systems and methods described herein advantageously obviate the need for mixing powdered material via multiple syringes, reduce waste and cost, maintain strict sterility, and improve the accuracy of administering pharmaceutical compositions, including by dispensing an exact amount of a mixture.

In one aspect, a carpule system is described herein. The carpule system comprises a carpule. In some embodiments, the carpule comprises a carpule body defining a first chamber and a second chamber, and a divider disposed between the first chamber and the second chamber, the divider being configured to isolate a first material in the first chamber from a second material in the second chamber. The system can further comprise a carpule mixing device configured to receive the carpule and displace a portion of the divider for mixing the first material with the second material to form a mixture. The system can further comprise a carpule syringe configured to receive the carpule and dispense an amount of the mixture, where the amount of the mixture is measurable via an audible sound and/or a tactile sensation provided by the carpule syringe.

In some embodiments, the mixture is disposed in a sealed mixing chamber having a volume that corresponds to volumes of the first and second chambers.

In further embodiments, the carpule mixing device in the carpule system is configured to pneumatically and/or mechanically rupture the divider.

In yet further embodiments, the carpule in the carpule system comprises a first external seal for sealing the first chamber and a second external seal for sealing the second chamber from the outside. The first and second external seals are disposed on opposing sides of the divider for maintaining an airtight seal inside the carpule body.

Moreover, in some embodiments, the first material in the first chamber is a liquid and the second material in the second chamber is a powder. The system liquid can be a solvent of the powder. The powder can be a pharmaceutical composition. In some embodiments, the powder comprises a botulinum toxin.

In another aspect, methods of forming a mixture using a carpule system are described herein. In some cases, such a method comprises providing a carpule, the carpule comprising a carpule body defining a first chamber and a second chamber and a divider disposed between the first chamber and the second chamber, the divider being configured to isolate a first material in the first chamber from a second material in the second chamber. The method further comprises displacing a portion of the divider, forming a mixture by mixing the first material with the second material, and inserting the carpule containing the mixture into a carpule syringe. Additionally, the carpule can be sealed on both a first end and a second end when the carpule is inserted into the carpule syringe.

In some embodiments, the method of forming a mixture further comprises dispensing an amount of the mixture from the carpule using the carpule syringe, wherein the amount being dispensed is measurable via an audible sound and/or a tactile sensation provided by the carpule syringe.

In yet another aspect, methods of administering a composition to a patient using a carpule system are described herein. In some cases, such a method comprises providing a carpule comprising a carpule body defining a first chamber and a second chamber and a divider disposed between the first chamber and the second chamber, the divider being configured to isolate a first material in the first chamber from a second material in the second chamber. The method further comprises displacing a portion of the divider to mix the first material and the second material to form the composition within the carpule body. The method further comprises inserting the carpule containing the composition in a carpule syringe and injecting the composition into the patient via the carpule syringe.

In some embodiments, the method further comprises storing the composition prior to injecting the composition into the patient and/or agitating the carpule to homogenize the composition prior to inserting the carpule into the syringe. The method may further comprise displacing a portion of the divider via pneumatically and/or mechanically rupturing the divider and/or measuring an exact amount of the composition injected into the patient via an audible sound and/or a tactile sensation provided by the carpule syringe.

These and other embodiments are described in more detail in the detailed description which follows.

### BRIEF DESCRIPTION OF THE FIGURES

FIG. 1 illustrates a perspective view of a carpule for use in a carpule system according to some embodiments described herein.

FIG. 2 schematically illustrates a carpule system and a method of using a carpule system according to some embodiments described herein.

FIG. 3 illustrates a method of forming a mixture using a carpule system according to some embodiments described herein.

FIG. 4 illustrates a method of administering a composition to a patient using a carpule system according to some embodiments described herein.

### DETAILED DESCRIPTION

Embodiments described herein can be understood more readily by reference to the following detailed description, examples, and figures. Elements, apparatus, systems, and/or methods described herein, however, are not limited to the specific embodiments presented in the detailed description, examples, and figures. It should be recognized that these embodiments are merely illustrative of the principles of the presently disclosed subject matter. Numerous modifications and adaptations will be readily apparent to those of skill in the art without departing from the scope of the presently disclosed subject matter set forth herein.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “from 5 to 10” or “5-10” should generally be considered to include the end points 5 and 10.

### I. Carpules

In one aspect, carpules, which may also be referred to as “carpule devices” are described herein. Carpules are configured for use in carpule systems as described hereinbelow, and may be used to house at least one medicinal drug and release the drug for delivery to a patient when actuated by a carpule syringe. As used herein, the term “carpule” refers to a structure that is insertable within a carpule syringe, or a portion thereof, the structure being configured to receive, house, and/or contain a material (e.g., a medicinal drug) until actuated by the carpule syringe. In some embodiments, actuation by the carpule syringe pressurizes portions of the carpule such that the material contained therein is discharged or expelled from the carpule and administered to a patient.

In some embodiments, carpules described herein are multi-chambered devices comprising a carpule body that defines at least a first chamber and a second chamber. More than two chambers can be provided in the carpule body, where desired. Each chamber may receive, as content, a given material, which may include a solid phase material (e.g., powder), a liquid phase material, a gas phase material and/or any combination thereof. In some embodiments, each chamber can contain the same material (e.g., having the same identity or composition) but may contain the same or different amounts, phases, volumes, and/or concentrations of the material. In other embodiments, different chambers can contain different materials (materials having differing compositions or identities), in the same or different amounts, volumes, and/or concentrations. The different materials may include materials having the same phase (e.g., both liquid phase materials), or different phases (e.g., a solid phase and a liquid phase).

For example and in some embodiments, the first chamber may contain a powder material and the second chamber may contain a liquid material. In other embodiments, the first chamber may contain a gas material and the second chamber may contain a liquid material. In further embodiments, the first chamber may contain a liquid material and the second chamber may also contain a liquid material, the types of liquids and/or the volumes of each respective liquid being the same or different. More than two chambers may be provided, where desired. Carpule described herein can comprise multiple chambers containing multiple materials, at least one of which is a liquid material. Upon mixing, the multiple materials can form a medication, drug, solution, fluid, and/or pharmaceutical composition.

In some embodiments, the contents or material contained within the first chamber of the carpule body can be isolated and/or separated from the content or material contained within the second chamber of the carpule body (and one or more other chambers of the carpule body, where provided), via at least one divider. The divider may include an internal divider that is disposed on or over or between one or more internal surfaces of the carpule body. In some embodiments, the divider is disposed between the first chamber and the second chamber of the carpule body for isolating the first material contained in the first chamber from the second material contained in the second chamber.

In some embodiments, the divider can comprise or be formed from any suitable material, for example, a flexible material, a frangible material, a brittle material, a penetrable material, a rupturable material, a soluble material, a disintegrable material, an organic material, an inorganic material, a degradable material, a metal, a perforated material, or any other type of material that can be physically, mechanically, chemically, thermally, electrically, or pneumatically ruptured or displaced to permit combination or mixing of the first and second materials in a manner described herein. The divider may at least partially flex against one or more surfaces of the carpule body providing spaces therethrough and/or otherwise at least partially open (e.g., via tearing, puncturing, ripping, breaking, displacing, etc.) such that the contents on opposing sides of the divider in opposing chambers can combine and mix upon displacing all or a portion of the divider.

In some embodiments, carpules described herein can further comprise a plurality of external seals configured to seal portions of the carpule body from the surrounding environment. In some embodiments, the external seals can comprise or be formed from any type sealing material such as a plastic, rubber, elastomer, metallic, or foam material. The external seals may prevent fluids, gases, and/or any other contaminants in the surrounding outside environment from entering the carpule body during mixing and/or dispensing a solution therefrom. In some embodiments, the contents of a respective chamber are sealed between the divider and a respective external seal. That is, prior to rupture or displacement, the divider can act as an internal seal within the carpule body to seal a given material in a respective chamber that forms between the divider and a respective external seal.

In some embodiments, carpules described herein comprise at least a first external seal for sealing the first chamber and at least a second external seal for sealing the second chamber. Each external seal may be at least partially disposed on or over external portions (e.g., external surfaces) of the carpule body for sealing the carpule body from the outside environment, thus avoiding introduction of air into the carpule body during preparing (e.g., mixing) and/or delivering the medicinal drug housed therein, the medicinal drug being a mixture of the contents in the first chamber and second chambers upon displacement of the internal divider. In some embodiments, each chamber is airtight and/or evacuated of gas. In other embodiments, a small amount of air or other gas may be present in one or more of the chambers so that the contents within each chamber may more readily mix upon displacing the divider. In some embodiments, when the divider is displaced, the carpule body remains sealed between the first and second external seals. The sealed carpule may then be inserted within a carpule syringe that administers the medicinal drug (or other material) contained within the sealed carpule to a patient (or other destination).

It is understood that carpules described herein can have any combination of properties or features described herein not inconsistent with the objectives of the instant disclosure.

### II. Carpule Systems

In a further aspect, carpule systems are described herein. Carpule systems described herein can utilize one or more multi-chambered carpules and advantageously introduce and mix the contents of each chamber within the carpule body to form a solution that has an extremely accurate concentration, without having to use multiple syringes and without introducing air or other contaminants into the solution and/or materials that form the solution. Carpule systems described herein are cost efficient by virtue of minimizing waste and human error associated with calculating, measuring, and/or preparing mixtures.

Turning now to specific components of carpule systems, carpule systems described herein include at least one carpule. Any carpule consistent with the above description in Section I can be provided and used in carpule systems described herein. In some embodiments, a carpule has a carpule body that defines at least a first chamber and a second chamber. Each chamber is configured to receive, as content, a given material. Discrete volumes or amounts of the same or different materials may be housed within discrete chambers of a given carpule. The materials contained in each chamber may include the same material, materials having a same phase (e.g., liquid phase materials, solid phase materials), different materials, and/or materials having a different phase. Any type and/or amount of material (e.g., a constituent of a medicinal drug or pharmaceutical composition) that is consistent with the present subject matter may be disposed within a given chamber of a multi-chambered carpule as described herein. Respective chambers of a multi-chambered carpule may include the same volume or different volumes, as desired.

In some embodiments, at least one divider is disposed between at least a first chamber and at least a second chamber of the carpule body for separating a first material contained in the first chamber from a second material contained in the second chamber. The divider can comprise or be formed from any suitable material that is consistent with the above description in Section I. For example, the divider can be formed from any type of material that can be physically, mechanically, chemically, thermally, electrically, or pneumatically ruptured or displaced to permit combination or mixing of the first and second materials in a manner described herein. In some embodiments, the divider comprises and/or is formed from a thin, penetrable member or membrane that allows contents on opposing sides thereof (e.g., in opposing chambers) to combine and mix upon displacing all or a portion of the member or membrane.

In certain embodiments, the divider can include one or more pre-stressed, thinned, perforated, or weakened areas that render the divider more susceptible to physically rupturing or tearing. For example and in some embodiments, the divider between chambers can comprise a plurality of perforations or any other type of localized areas having a minimum thickness that are not fully punctured, but are thin enough so that tearing, puncturing, or physical displacement of the divider is more readily obtained with minimal pressure or force. In some embodiments, the divider between the at least two chambers includes a thin layer of foil (e.g., a metal or a plastic foil), with or without preformed perforations.

In certain embodiments, the divider can have an average thickness of between about 50 nanometers (nm) to about 5 millimeters (mm). For example and in some embodiments, a divider disposed in carpules described herein can have an average thickness of about 50 nm or more, 100 nm or more, 500 nm or more, 1 μm or more, 100 μm or more, 500 μm or more, 1 mm or more, 2 mm or more, or 5 mm or more. In some cases, the divider has an average thickness of about 50 nm to 500 nm, 100 nm to 1 m, 50-100 m, 50-250 m, 100-500 m, 500 μm-1 mm, 0.1 mm-5.0 mm, 0.1-0.5 mm, 0.1-0.25 mm, 0.5-5.0 mm, 1.0-5.0 mm, or more than 5 mm. Additionally, the sectional shape, size (e.g., diameter, length, width, and/or thickness), and/or quantity of dividers can be selected based on the size, shape, contents, and/or volume of the carpule body and/or the size, shape, contents, volume, and/or quantity of the carpule chambers, and/or based on the materials to be mixed. The divider can include or be formed from any material capable of isolating or separating (e.g., physically, thermally, and/or electrically) two or more chambers from one another in a carpule body. In some instances, the divider is disposed on or over or between internal portions (e.g., surfaces) of the carpule body and the carpule body can comprise or be formed from a glass, plastic, polymeric, or metallic material.

In some embodiments, the divider extends from portions of an inner surface of the carpule and/or adheres to an inner surface of the carpule via adhesive, thermal bonding material, epoxy, or glue. In other embodiments, the divider seals one chamber and adheres to an upper surface (e.g., an upper rim) of a first chamber, and a second chamber can be provided over and/or on an upper surface of the divider. Thus, the divider may adhere to opposing surfaces of each respective chamber to which it is adjacent and/or disposed between. Dividers can comprise a sectional shape that is a square, curved, rounded, circular, or non-circular. Any size, shape, location, and/or material associated with dividers described herein is contemplated, and can be provided.

As noted in Section I above, carpules utilized by carpule systems described herein can include one or more external seals disposed on or over the carpule body for sealing the second chamber from air and/or contaminants contained in the outside environment. The first and second external seals can be disposed on opposing sides of the divider for maintaining an airtight seal inside the carpule body and a respective chamber of the carpule body. In some embodiments, at least one of the first and second seals can be movable, displaceable, and/or slidable within the carpule body upon application of pressure (e.g., depressing) to the respective seal by a portion of a carpule syringe or carpule mixing device (e.g., a plunger or header). As it moves, slides, or is displaced, the external seal may increase pressure on or within the carpule body and expel the mixture from the carpule syringe.

Carpule systems described herein further comprise a plurality of chambers. Each chamber can comprise the same volume, or each chamber can comprise a different volume. Where more than two chambers are provided, any combination of same and/or different volumes of chambers can be provided. In some embodiments, each of the plurality of chambers is coaxially aligned. In other embodiments, each of the plurality of chambers is elongated having an elongated axis that is substantially parallel to an elongated axis of one or more other chambers. Any size, shape, and/or orientation of chambers is contemplated.

In some embodiments, one or more of the chambers within a carpule utilized by carpule systems described herein has a chamber volume of between about 0.001 milliliters (mL) and 5 mL. For example, one or more of the carpule chambers can comprise a volume of about 0.001 mL or more, 0.002 mL or more, 0.005 mL or more, 0.01 mL or more, 0.025 mL or more, 0.04 mL or more, 0.05 mL or more, 0.1 mL or more, 0.25 mL or more, 0.5 mL or more, 1 mL or more, 2 mL or more, 3 mL or more, or more than 4 mL. In some cases, one or more of the chambers within a carpule utilized by carpule systems described herein has a chamber volume of about 0.001-0.005 mL, 0.001-0.008 mL, 0.01-0.05 mL, 0.025-0.05 mL, 0.1-0.5 mL, 0.1-1.0 mL, 0.1-2 mL, 1-2 mL, 1-5 mL, or 1-10 mL. Any volume of chamber within a given carpule utilized by carpule systems described herein can be provided.

In some embodiments, at least one chamber can contain a powder and at least one chamber can contain a liquid. A powder/liquid ratio may be provided by adjusting the respective powder/liquid chamber sizes and/or volumes within a respective carpule.

In some embodiments, when one or more of the dividers between adjacent chambers becomes displaced, the overall (i.e., undivided, total) volume of the carpule can range between about 0.025 mL and 10 mL. In certain embodiments, the overall volume of the carpule can be 0.025 mL or more, 0.05 mL or more, 0.1 mL or more, 0.25 mL or more, 0.5 mL or more, 1 mL or more, 2 mL or more, 3 mL or more, 4 mL or more, 5 mL or more, or more than 6 mL. Upon displacing the divider, or a portion thereof, contents within adjacent chambers can mix within the overall sealed volume of the carpule without introduction of air or other contaminants. In some cases, overall volume of the carpule is about 0.5-1 mL, 0.5-0.8 mL, 0.5-2 mL, 0.5-3 mL, 1-1.5 mL, 1-1.8 mL, 1.5-2 mL, 1.5-3 mL, 1.8-3 mL, 2-5 mL, 3-6 mL, or 5-10 mL. Carpules utilized by carpule systems described herein are not limited to any particular volume, and any volume not inconsistent with the present subject matter may be used.

In some embodiments, carpule systems described herein utilize at least a first chamber containing a powder and at least a second chamber containing a liquid. Any desired powder/liquid ratio of mixture may be prepared and dispensed from the carpule via adjusting the respective powder/liquid chamber size and/or volume. For example, mixtures having a powder/liquid ratio of at least about 0.1, 0.125, 0.25, 0.5, or 1, by volume, may be provided. The powder chamber may be about 1/10, ⅛, ¼, or ½ the size, by volume, of the liquid chamber. Any ratio between a powder/liquid may be provided, where at least one powder and one liquid are provided.

In some embodiments, at least a first chamber of the multi-chambered carpule has a powdered material stored therein, such as a soluble powdered medicine or pharmaceutical composition. Powdered medicines can comprise, for example, an anesthetic powder, an analgesic powder, a pharmaceutical powder, a powder comprising an injectable neurotoxin such as a botulinum toxin, or any other pharmaceutical or medicinal composition, material, or substance. In an exemplary embodiment, at least one chamber of the carpule comprises a volume of powder corresponding to one or more units or doses of a BOTOX® powder, which is manufactured by Allergan, PLC, having headquarters in Irvine, Calif. In other cases, at least one chamber of the carpule comprises a volume of powder corresponding to one or more units or doses of DYSPORT® powder (Medicis Pharmaceutical), or XEOMIN® powder (Merz Pharma).

In further embodiments, at least a second chamber of the multi-chambered carpule has a liquid stored therein. The liquid can include a predetermined (i.e., premeasured) amount or volume of a diluent or solvent of the powder. The liquid can be used to reconstitute a predetermined (i.e., premeasured) amount, dose, volume, or unit of a powdered material contained in at least a first chamber of the carpule body prior to injection into a patient (or otherwise dispensing the material). Upon removing, rupturing, or displacing the divider (or portion thereof) contents in the first chamber (e.g., the powder) mix with the contents in the second chamber (e.g., the liquid). The liquid and powder can mix and form a volume of a liquid phase composition that occupies the sealed carpule volume between the first and second seals prior to injection in the patient (or other dispensing of the mixture). In cases where the liquid is a solvent of the powder, the powder can dissolve upon displacing the divider and introducing the liquid to the powder to form a liquid phase material, such as a liquid medicine and/or a liquid pharmaceutical composition that can be administered to a patient. In some embodiments, the liquid within a multi-chambered carpule can comprise or be formed from saline, water, alcohol, or an organic solvent. Any liquid not inconsistent with the objections of the present subject matter can be provided.

Notably and in some embodiments, the liquid and powder material in the respective first and second chambers can be premeasured and individually sealed such that mixing of the respective contents in a desired ratio occurs automatically within the sealed carpule upon removing, rupturing, and/or displacing a portion of the divider. As different amounts (e.g., volumes) of powder and liquid can be provided in different carpule chambers, the carpule systems described herein can provide very precise and accurate volumes, dosages, ratios, units, and/or concentrations of medicinal compositions, pharmaceutical compositions, and/or solutions, without having to introduce air and/or contaminants, and/or without under- and/or over-diluting the powdered material. Carpule systems described herein can advantageously combine a predetermined amount (i.e., a precise or controlled volume) of powdered material with a predetermined amount (i.e., a precise or controlled volume) of liquid (e.g., solvent or diluent) upon removal of a divider within the carpule for forming pharmaceutical solutions or compositions having any desired concentration. Removal of the divider allows the liquid and powder to mix and form a mixture in a sealed mixing chamber with a volume corresponding to volumes of the first and second chambers.

Carpule systems described herein can further comprise an optional carpule mixing device. The carpule mixing device is configured to receive a multi-chambered carpule as described herein, and displace a portion of the carpule divider for mixing a first material in the first carpule chamber with a second material in the second carpule chamber to form a mixture. The mixture can include a solution, a medicine, or a pharmaceutical composition that can be administered to a patient.

In some embodiments, the carpule mixing device comprises a housing having a lid. The housing is configured to receive one or more carpules and to displace dividers within the one or more carpules upon depressing the lid. The housing may contain one or more slots, racks, depressions, or cavities sized and/or shaped to receive one or more carpules. The size and/or shape of the cavities can be adjustable, so that one carpule mixing device is configured to receive any size (e.g., length, diameter, width) of carpule.

In some embodiments, depressing the lid of the carpule mixing device applies minimum force or pressure to one or more portions of the carpule(s) contained therein, which displaces or ruptures one or more dividers disposed within each carpule. In other embodiments, depressing the lid can create a pressure differential that physically, mechanically, or pneumatically ruptures or displaces carpule dividers. Depressing the lid of the carpule mixing device may also chemically, thermally, or electrically rupture (e.g., permeate, displace, etc.) a portion of one or more dividers disposed within each carpule, where desired. In some embodiments, depressing the lid of the carpule mixing device causes a mechanical piercing member (e.g., mechanical arm) to pivot, move, or otherwise change location for mechanically rupturing the divider. In further embodiments, depressing the lid causes light to degrade the divider, for example, UV light may irradiate the divider and cause the divider to degrade or rupture if the divider is not UV-stable. Any method of rupturing or displacing a divider is contemplated.

In yet further embodiments, depressing the lid of the carpule mixing device can trigger a change in internal attributes of the carpule mixing device. As the physical, chemical, mechanical, electrical, pneumatic, and/or thermal attributes within the carpule mixing device change, the divider may become ruptured and/or displaced. When the divider is removed, ruptured, flexed, or displaced, the chamber contents isolated by the divider can freely interact and mix within a carpule volume defined between at least a first and a second external seal.

Still referring to carpule systems and in some embodiments, the carpule mixing device can optionally agitate the carpules housed therein after rupturing or permeating the respective dividers of the one or more carpules, thereby providing a uniform and/or homogenized mixture of the powder and liquid constituents within respective carpule chambers. The carpule mixing device may include a timer for timed mixing, where desired. Thus, in some cases, the carpule mixing device can “activate” a carpule (by permitting mixing of components of separate carpule chambers) and also agitate the carpule to provide a substantially homogeneous mixture. In this manner, the carpule mixing device can prepare a multi-chambered carpule described herein for use, such as use for treating a patient.

Carpule systems described herein can further comprise a carpule syringe configured to receive the carpule, including upon removal from the carpule mixing device. The carpule syringe can be used to dispense gradual and/or incremental amounts of the mixture from the carpule. The incremental amounts of the mixture dispensed from the carpule may be identical increments or variable increments, where desired. In some embodiments, the volume of the mixture within the carpule corresponds to one or more units or doses (including an integral or half-integral number of units or doses) of a medical drug, solution, or pharmaceutical composition. In other embodiments, an incremental amount of the mixture being dispensed from the carpule corresponds to one unit or dose of the medical drug, solution, or pharmaceutical composition, for example, in cases where the carpule contains more than one unit or dose.

In some embodiments, the entire amount of the mixture may be dispensed from the carpule and administered to only one location on a patient's body (e.g., all of the mixture may be dispensed to a location in a patient's jaw, forehead, etc.) or each incremental amount of the mixture may be dispensed from the carpule and administered to different locations on a patient's body (e.g., various locations on a patient's scalp, forehead, face, etc.).

In certain embodiments, the carpule containing the mixture may be optionally stored prior to insertion into a carpule syringe and/or injection of the mixture into a patient. Where stored, the carpule may be stored at room temperature (e.g., about 70° F. or 21° C.), or temperatures less than room temperature. In some embodiments, the carpule containing the mixture may be stored and shipped to multiple locations (e.g., doctor offices, dentist offices, hospitals, drug stores, pharmacies, etc.) prior to injection of the mixture into a patient. In some embodiments, the carpule containing the mixture may be stored in a cooled storage structure, such as a refrigerator or freezer. For example and in some embodiments, the carpule containing the mixture may be stored for at least one minute, at least 10 minutes, at least 20 minutes, at least one hour, at least 6 hours, at least 24 hours, at least two days, at least 5 days, at least 7 days, at least 2 weeks, at least 3 weeks, at least 4 weeks, at least 2 months, at least 6 months, at least 12 months, at least 18 months, or more than 20 months.

Notably, the amount of the mixture being dispensed from the carpule syringe can be measurable via an audible sound and/or a tactile sensation provided by the carpule syringe. The increments at which the audible sound and/or tactile sensation are perceived can be adjusted to correspond to any desired volume, amount, unit, or dose of dispensed mixture. The audible sound and/or tactile sensation can be adjusted for corresponding to various graduated or discrete amounts of a mixture to be dispensed, and the graduated or discrete amounts may be identical (e.g., equal) amounts or unequal amounts, where desired. In some embodiments, each discrete amount of the mixture to be dispensed corresponds to one unit or dose of a medication or pharmaceutical composition, or a fraction of one unit or dose of the medication or pharmaceutical composition (such as one-half of a unit or dose).

Measuring an amount of mixture being dispensed form a carpule syringe via audible and/or tactile sensations advantageously allows persons administering the mixture to readily recognize and/or easily realize the amount, volume, dose and/or number of units of the mixture being dispensed to a patient without having to visually verify or mentally calculate such. Thus, precise amounts, doses, volumes, and/or units of the mixture dispensed from the carpule may be dispensed to different regions or areas of a patient's body, the combined amount of which is measurable via an audibly perceivable signal (e.g., a click or beep) and/or a tactilely perceivable signal (e.g., a twitch, depression, a change in resistance, or a punch sensation). After a desired graduation is selected, portions of the carpule syringe can be locked in a desired position via a locking mechanism to prevent any undesired dispensing of the mixture from the carpule, such as may be caused by a pressure change that occurs when a needle of the syringe is removed from tissue and exposed to the external environment (e.g., air) instead.

In some embodiments, carpule syringes utilized in carpule systems described herein include a hollow barrel or housing that receives a carpule containing a mixture (e.g., pharmaceutical composition) therein. The syringe may include a handle or gripping portion connected to a plunger. The plunger may depress a portion of the carpule and apply a pressure thereto for dispensing gradual or discrete amounts of the mixture from the carpule. As the plunger expels the mixture from the carpule, it may trigger the audible and/or tactile sensations indicative of an amount of the mixture being dispensed (e.g., injected to a patient).

In some embodiments, the barrel of the syringe comprises readable graduations extending along a length of the barrel. The graduations can correspond to units, doses, volumes and/or fractions thereof that correspond to the amount of the mixture to be dispensed and measured as the plunger moves to inject and/or otherwise administer the mixture to a patient. The graduations may be adjusted to any desired increment or measurement via moving the barrel, rotating the barrel, sliding the barrel, telescoping the barrel, using a key, a tool, etc., for adjusting portions of the syringe that will audibly or tactilely indicate a graduated amount of the mixture being dispensed as the plunger moves through the barrel. The respective graduation increments may reflect common volume units used in administering injections, for example, the syringe may audibly or tactilely indicate graduation increments of 0.025 mL, 0.04 mL, and 0.05 mL, or any other desired increment based on the type of mixture being dispensed.

In response to dispensing a metered amount of the mixture to a patient, the carpule syringe may emit an audible sound (e.g., a click or beep) and/or a tactile sensation (e.g., an increase in resistance, a hard stop, a pause, etc.) that indicates the amount of the mixture being injected to a patient. For example, the carpule syringe barrel may optionally include equidistant or non-equidistant formations, such as protrusions or indentations. The formations are configured to provide an audible sound and/or a tactile sensation as a plunger of the carpule syringe encounters each formation. The audible sound and/or tactile sensation is indicative of the amount of mixture being dispensed. Any type of carpule metering device or mechanism may be provided for injecting known amounts of the mixture to the patient.

For example and in some embodiments, carpule syringes utilized in carpule systems described herein may include at least one external and/or internal mechanism configured to dispense an incremental or metered amount of the carpule mixture to a patient. For example and in some embodiments, carpule syringes described herein may be provided with a pump or pressure mechanism configured to apply a force and/or suction to the syringe plunger to drive the mixture into the patient and at a controlled rate, increment, or graduation. As the pump mechanism applies pressure to the plunger, the syringe may emit an audible and/or tactile sensation in response (e.g., a beep, click, or tactile perceptible increase in resistance), which is indicative of an amount of the mixture being dispensed (e.g., injected to a patient).

Notably, powders and reconstituting liquids can be pre-packaged in multi-chambered carpules, precisely mixed in a sealed volume, and then injected into a patient via a carpule syringe in gradual or incremental or “quantized” amounts that can be easily detected or measured via audible or tactile sensations as described herein. Carpule systems herein advantageously dispense pre-selected and known units or doses of a mixture into a patient, without having to visually verify the amount being dispensed.

It is understood that carpules or carpule systems described herein can have any combination of properties or features described herein not inconsistent with the objectives of the instant disclosure.

III. Methods of Forming and/or Dispensing a Mixture Using a Carpule System

In yet another aspect, methods of forming and/or dispensing a mixture using a carpule system are provided. In some embodiments, a method comprises providing any type of carpule as described in Sections I and II above. For example, the carpule can comprise a carpule body defining a plurality of chambers containing discrete amounts of material that mix upon displacement of a divider. The divider can be disposed between two or more chambers to isolate material in one chamber from material in another chamber. Notably, one material can comprise a powder, such as a powdered medicine, and the other material can comprise a liquid. In some embodiments, the liquid is a solvent of the powder, such that displacing the divider causes the powder and liquid to mix and form a mixture or composition. The mixture or composition can comprise a pharmaceutical composition, optionally comprising a botulinum toxin including but not limited to BOTOX®, DYSPORT®, or XEOMIN®

In some embodiments, a method of forming a mixture further comprises displacing a portion of the divider. The term “displacing” used in connection with the divider, refers to rupturing the divider and/or portions thereof, piercing the divider and/or portions thereof, flexing the divider and/or portions thereof, penetrating the divider and/or portions thereof, or removing the divider and/or portions thereof in such a manner that permits physical mixing of materials in the two or more carpule chambers.

In some embodiments, displacing the divider comprises placing the divided carpule within a housing of a carpule mixing device and depressing or closing a lid of the carpule mixing device over the carpule(s) housed therein. Depressing or closing the lid of the mixing device can trigger a change in pressure, heat, electrical attributes, lighting attributes (e.g., introduction of UV light), chemical attributes, and/or a location of a mechanical piercing member within the mixing device and/or with respect to the carpule housed therein as described above. The change in pressure, heat, electrical, light, or chemical attributes and/or the location of a mechanical piercing member can respectively pneumatically, thermally, electrically, optically, chemically, and/or mechanically rupture or degrade the divider. Rupturing the divider removes the divider, or portions of the divider, between adjacent chambers, thus, the contents of the respective chambers can freely interact and mix within a carpule volume defined between at least a first and a second external seal.

In some embodiments, a method of forming a mixture further comprises forming a mixture by mixing the first material with the second material. Mixing the first material with the second material can comprise interacting the first material (e.g., powder) with a second material (e.g., liquid), which is a solvent of the first material.

In some embodiments, mixing the first and second materials can optionally comprise agitating the carpule via the carpule mixing device to homogenize the mixture. The mixture may be mixed for any period of time including but not limited to between about 3 seconds and 30 minutes. For example, the mixture may be mixed for about 3 seconds or more, 5 seconds or more, 10 seconds or more, 30 seconds or more, 1 minute or more, 2 minutes or more, 5 minutes or more, 10 minutes or more, or more than 15 minutes.

The carpule and mixture may optionally be stored prior to dispensing the mixture (e.g., by administering the mixture and/or injecting the mixture into a patient). When stored, the mixture may be stored for anywhere from between one minute and 2 years. For example and in some embodiments, the mixture may be stored for at least one minute, at least 10 minutes, at least 20 minutes, at least one hour, at least 6 hours, at least 24 hours, at least two days, at least 5 days, at least 7 days, at least 2 weeks, at least 3 weeks, at least 4 weeks, at least 2 months, at least 6 months, at least 12 months, at least 18 months, or more than 20 months.

In some embodiments, the method further comprises inserting the carpule containing the mixture into a carpule syringe. The carpule can be sealed via seals on both a first end and a second end via respective first and second external seals prior to being inserted into the carpule syringe as described above. In some embodiments, the carpule is inserted into a barrel of a carpule syringe. The barrel can optionally comprise adjustable graduations so that known, measureable graduations of the mixture can be dispensed as described in Section II above.

In some embodiments, a method further comprises dispensing an amount of the mixture from the carpule using the carpule syringe, where the amount being dispensed is measurable via an audible sound and/or a tactile sensation provided by the carpule syringe as described in Section II above. For example, the carpule syringe barrel may include equidistant or non-equidistant protrusions or indentations configured to provide an audible sound and/or a tactile sensation indicative of the amount of mixture being dispensed.

It should further be understood that a method of forming and/or dispensing a mixture using a carpule system as described herein can have any combination of properties or features described herein not inconsistent with the objectives of the present disclosure.

### IV. Methods of Using a Carpule System to Administer a Composition to a Patient

In yet a further aspect, methods of administering a composition to a patient are provided. The composition can comprises any type of mixture administered from any type of carpule and/or carpule system consistent with Sections I to III above. In some embodiments, the method includes providing a carpule having a carpule body that defines at least two chambers, where a divider is disposed between the at least two chambers. The divider isolates material in one chamber from material in at least one other chamber. The first and second materials can comprise any of the materials described hereinabove in Sections I to III above.

A method can further comprise displacing a portion of the divider to mix the first material in the first chamber with the second material in the second chamber to form the composition within the carpule body. The carpule body can be sealed from the surrounding environment via one or more external seals. The divider can become displaced upon depressing or closing a lid of a carpule mixing device over one or more multi-chambered carpules housed therein. Depressing or closing the lid of the mixing device can trigger a change in internal attributes of the carpule mixing device. As the physical, chemical, mechanical, electrical, pneumatic, and/or thermal attributes within the carpule mixing device change, the divider may become ruptured and/or displaced. When the divider is removed, ruptured, flexed, or displaced, the chamber contents isolated by the divider can freely interact and mix within a carpule volume defined between at least a first and a second external seal.

In some embodiments, the method further comprises inserting the carpule containing the mixed composition in a carpule syringe and injecting the composition into the patient via the carpule syringe. The amount of the composition injected into the patient can be measured via an audible sound and/or a tactile sensation provided by the carpule syringe. A barrel of the carpule syringe may include a plurality of equidistant or non-equidistant protrusions or indentations that correspond to graduated amounts of a composition to be injected into the patient. As the plunger passes over the protrusions or indentations, the carpule syringe can emit a perceptible audible sound and/or tactile sensation indicative of the amount of the composition being injected into the patient.

In some embodiments, the composition can be stored in the sealed carpule prior to injecting the patient. The composition may be stored for any length of time as noted in Sections II and III above, for example, in a refrigerated or non-refrigerated storage unit. In some embodiments, the composition can be stored in the sealed carpule and shipped prior to injection in a patient for treating the patient.

In some embodiments, administering a composition to a patient further comprises inserting the carpule having the mixture into the carpule syringe and depressing a portion of the carpule syringe to expel the mixture from the carpule. An injecting member, such as a needle or nozzle, can be inserted on, over, against, and/or into a patient such that the mixture is injected on, over, against, and/or into the patient and thus administered thereto. In some embodiments “administering” the mixture refers to injecting the mixture into a patient's body and/or topically applying the mixture to a patient's body.

Moreover, in some cases, administering a composition to a patient comprises administering the composition to more than one location or region of the patient's body. For example, in some instances, a method described herein comprises administering the composition to a first location of the patient's body and to a second location of the patient's body, the first and second locations differing from one another. For instance, in some embodiments, the first and second locations are differing locations on a forehead of the patient, or a location on a forehead of the patient and a location on a jaw of the patient. Moreover, the composition can be administered to the first and second locations in a sequential manner. Further, in some such cases, the composition is administered to each sequential location in a manner described herein, such as in a manner guided by an auditory or tactile sensation for indicating to an administrator (e.g., a doctor) when a desired or correct amount of the composition has been administered to the specific location. It is further to be understood that a method described herein can comprise administering a composition to more than two locations of a patient's body in such a manner. In general, a composition described herein can be administered to n differing locations of the patient's body, wherein n can be an integer between 1 and 100, between 1 and 50, between 1 and 20, or between 1 and 10. Moreover, a composition described herein can be administered to n locations of the patient's body in n amounts, where the n amounts can be the same or different, depending on a desired composition delivery profile. For example, in some cases, a first amount of a composition is administered to a first location of a patient's body (e.g., a first location on the patient's forehead), a second amount of the composition is administered to a second location of a patient's body (e.g., a second location on the patient's forehead differing from the first location), a third amount of the composition is administered to a third location of a patient's body (e.g., a third location on the patient's forehead differing from the first and second locations), and a fourth amount of the composition is administered to a fourth location of a patient's body (e.g., a fourth location on the patient's forehead differing from the first, second, and third locations), wherein the first, second, third, and fourth amounts are the same or different. Other combinations are also possible, as understood by one of ordinary skill in the art.

It is understood that a method of administering a composition to a patient using a carpule system as described herein can have any combination of properties or features described herein not inconsistent with the objectives of the present disclosure.

Some exemplary embodiments described herein are further illustrated in the following non-limiting examples.

### Example 1

### Carpule and Carpule System

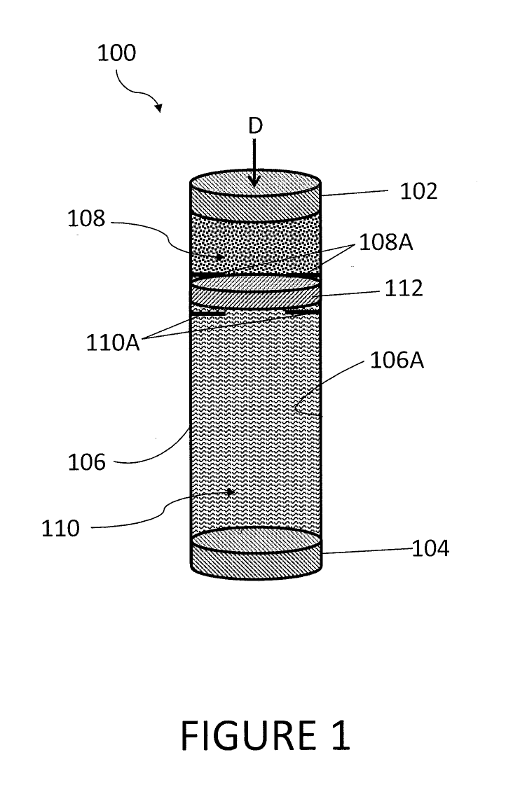

FIG. 1 schematically illustrates an exemplary embodiment of a carpule 100, which can be utilized by carpule systems described herein. Carpule 100 comprises a first end having a first external seal 102 and a second end having a second external seal 104. Respective first and second external seals 102 and 104 can seal a carpule body 106 from the outside. First and second seals 102 and 104 can comprise or be formed from any type sealing material not inconsistent with the present subject matter, such as plastic, rubber, elastomer, metal, or foam.

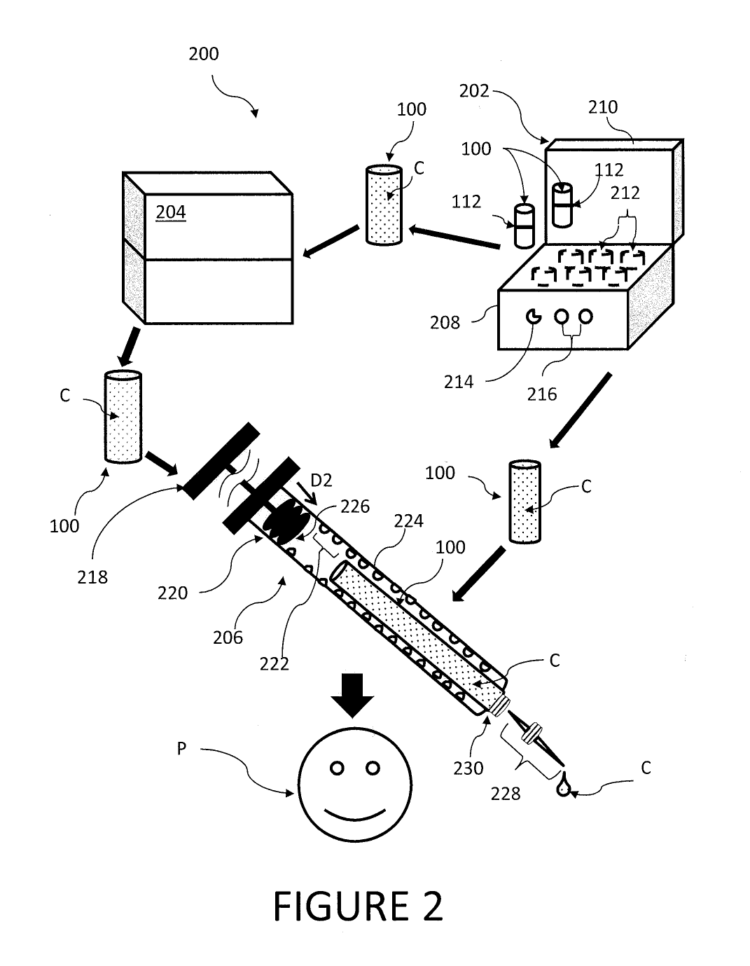

In some embodiments, first seal 102 is movable (e.g., slidable) in a direction D with respect to carpule body 106, for example, upon depression of and/or pressure applied by a plunger, header, or other member of a carpule mixing device (e.g., 202, FIG. 2) or a carpule syringe (e.g., 206, FIG. 2). Second seal 104 can be punctured, for example, by a tip or needle assembly (e.g., 228, FIG. 2) of a carpule syringe so that the mixture or composition stored in carpule body 106 can be expelled from the carpule via the carpule syringe.

Carpule body 106 defines at least a first chamber 108 and a second chamber 110. Although only two chambers are illustrated in carpule 100, more than two chambers may be defined and/or formed in carpule body 106. As FIG. 1 illustrates, first and second chambers 108 and 110 are divided by a divider 112. Contents in first and second chambers 108 and 110 are also divided and/or isolated by divider 112.

In an exemplary embodiment, first chamber 108 contains a volume of powder material and second chamber 110 contains a volume of liquid. The powder contained in first chamber 108 can comprise, for example, a powder medicine, an anesthetic powder, an analgesic powder, a pharmaceutical powder, or a powder comprising a botulinum toxin such as BOTOX®. The powder contained in first chamber 108 can be a soluble powder. The liquid contained in second chamber 110 can dissolve the powder in first chamber 108, and can be a solvent of the powder, such as saline.

As FIG. 1 further illustrates, divider 112 is disposed between first chamber 108 and second chamber 110, or portions thereof. In some embodiments, divider 112 is positioned between opposing planar surfaces 108A and 110A (e.g., rims) of the first and second chambers 108 and 110. In other embodiments, divider 112 adheres to and/or is positioned between one or more inner surfaces 106A of carpule body 106. Any position or location of divider 112 that is between respective first and second chambers 108 and 110 is contemplated.

Carpules such as those described in this Example offer many advantages compared to other carpules. For example, powder can be reconstituted within a sealed carpule which prevents introduction of air and/or contaminants to the composition. Moreover, the mixture can be sealed and stored prior to injecting the patient.

FIG. 2 illustrates various aspects of an exemplary carpule system 200. Carpule system 200 comprises one or more carpules 100, a carpule mixing device 202, an optional carpule storage device 204, and a carpule syringe 206.

In an exemplary embodiment, carpule mixing device 202 comprises a carpule housing 208 and a lid 210. Carpule housing 208 is configured to receive and retain one or more carpules 100 therein, for example, in some embodiments, within one or more retaining structures, slots, or cavities 212. In some embodiments, housing 208 is configured to receive and retain a plurality of carpules 100 therein, each carpule being sealed by at least one divider 112.

Carpule mixing device 202 is configured to displace a divider 112 of a respective carpule 100. For example, after one or more carpules 100 are loaded within housing 208, lid 210 can be depressed. Closing and/or depressing lid 210 triggers mixing device 202 to apply a pressure (e.g., via a mechanical arm or piercing member, not shown) to create a pressure differential, or otherwise change an attribute associated with housing 208 that causes divider 112 to become removed, displaced, or ruptured. Attributes of mixing device 210 may be controlled via adjusting a timer 214 and/or any number of adjustable controls 216 (e.g., for controlling pressure attributes, mechanical attributes, mechanical piercing members (not shown), electrical attributes, chemical attributes, or lighting attributes) that assist in displacing divider 112. After divider 112 is displaced, mixing device 202 may optionally agitate the carpules until mixed, and the agitation may optionally be timed via timer 214.

After divider 112 is removed or displaced, the carpules 100 may be removed. As FIG. 2 illustrates, after processing at carpule mixing device 202, each carpule 100 contains a fully mixed composition C. Carpule 100 is sealed via first and second seals (FIG. 1) and composition C can occupy any volume as defined between first and second seals (FIG. 1).

Still referring to FIG. 2 and in some embodiments, carpule 100 can optionally be stored in a storage unit or storage device 204 prior to composition C being administered to a patient or otherwise dispensed from sealed carpule 100. Carpule 100 can be stored for any amount of time prior to administering composition C to a patient. In some embodiments, storage device 204 is a storage structure, such as a cabinet, freezer, or a refrigerator configured storing a plurality of carpules 100 at about room temperature (i.e., about 70° F.) or at temperatures less than room temperature. For example, carpules 100 can be stored in device 204 at a temperature of about 68° F. or less, about 60° F. or less, about 55° F. or less, about 45° F. or less, or less than 30 OF.

As FIG. 2 further illustrates, system 200 can comprise carpule syringe 206. Composition C can be dispensed or expelled from carpule 100 via carpule syringe 206. Syringe 206 can comprise a gripping portion 218 attached to a plunger 220. Plunger 220 moves in response to extension or retraction of gripping portion 218 with respect to a barrel 224 of carpule syringe 206.

As plunger 220 moves along a length of barrel 224 in a direction D2, it encounters one or more incremental formations 222 that can take the form of indentations or protrusions. Each of the formations 222 can be spaced apart by equal and/or unequal distances. Each of the formations 222 corresponds to a graduated, discrete amount of composition C to be dispensed from carpule 100. As plunger 220 encounters (e.g., frictionally engages) or otherwise interacts with formations 222, carpule syringe 206 can emit an audible sound and/or a tactile sensation that is used to measure an amount of composition C being dispensed to a patient P.

For exemplary purposes only, a graduated barrel 224 is illustrated is illustrated in FIG. 2. However, as persons having skill in the art will appreciate, any other type of carpule syringe metering/actuating device may be provided for dispensing a desired amount and/or graduated amounts of a carpule mixture to a patient. That is, in some embodiments, barrel 224 is devoid of formations 222. Carpule syringe 206 may instead be fitted with an external or internal syringe actuating device (not shown) such as a syringe pump, receptacle device, or actuating member configured to apply an external force or suction to actuate plunger 220 and dispense a metered amount of composition C to a patient. In response to dispensing a metered amount, carpule syringe 206 may emit an audible sound (e.g., a click or beep) or a tactile sensation (e.g., an increase in resistance, a hard stop, a pause, etc.) that indicates the amount of composition C being injected to a patient P. Any type of carpule metering device or mechanism may be provided for injecting known amounts of composition C to a patient P. Additionally, the audible sound can be provided by any device and in any manner not inconsistent with the objectives of the present disclosure. For instance, in some cases, an electronic speaker or other device for producing an auditory signal can be directly or indirectly attached to or otherwise coupled to the carpule syringe 206 (or to another element of a system described herein). Further, in some embodiments, the electronic speaker or other device for producing an auditory signal is also directly or indirectly attached to a microprocessor or computer that actuates or controls the electronic speaker or other device.

According to FIG. 2, carpule 100 contains a fully mixed composition C, which is inserted within an opening 226 of syringe 206. Plunger 220 depresses against an end of carpule 100 and displaces the seal (e.g., 102, FIG. 1) thereby expelling composition C from an opposing end 230 of syringe 206 that is distal to gripping portion 218. The distal end 230 of syringe 206 can include a tip or needle assembly 228 that attaches the syringe 206 via a threaded connection, a connector, a lock, a frictional engagement, or a pin. Needle assembly 228 can comprise opposing ends, one end being configured to pierce the sealed end (e.g., 104) of carpule 100, and an opposing end being configured to penetrate an injection site of a patient P. Upon insertion of a portion of needle assembly 228 into patient P, composition C is then injected into patient P.

Carpule systems such as those described in this Example offer many advantages compared to other systems. For example, powder can be reconstituted and uniformly mixed within a sealed carpule, the mixture being suitable for injection into a patient at graduated amounts that are measureable via an audible sound and/or tactile sensation without having to visually verify or manually calculate the injected amount.

### Example 2

### Mixing and Administering Compositions Using a Carpule System

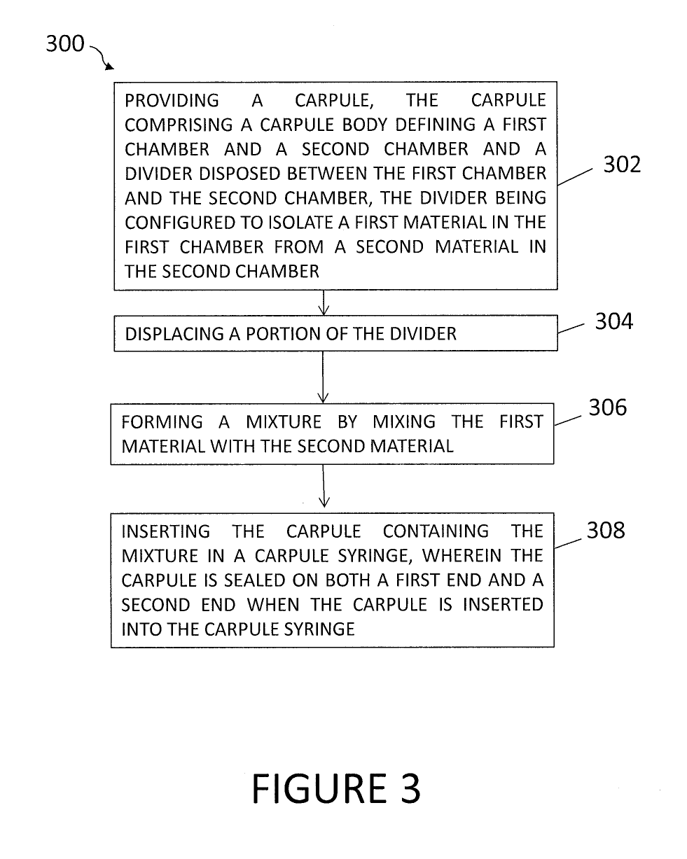

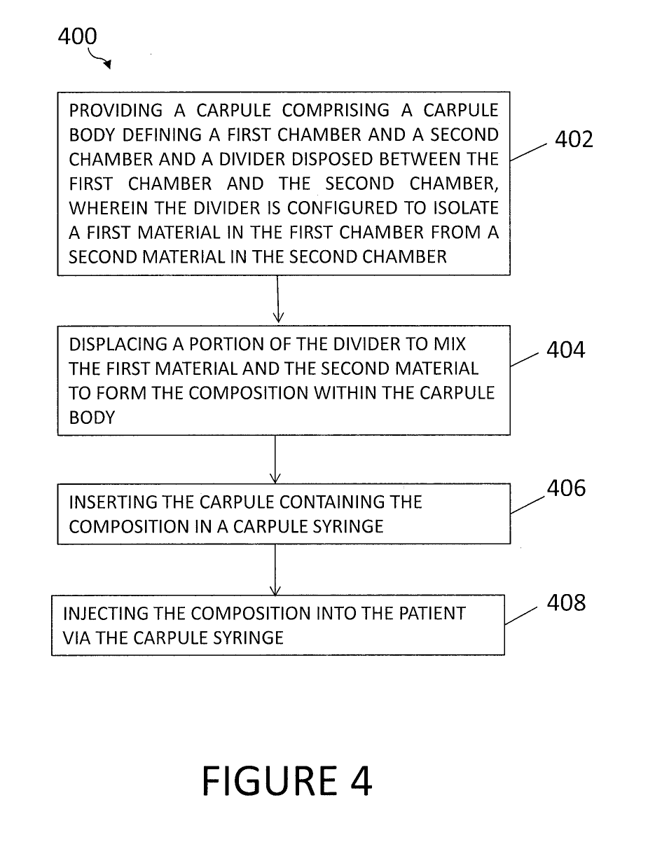

FIGS. 3 and 4 illustrate exemplary methods of mixing and administering a composition using a carpule system, including but not limited to mixing and administering a pharmaceutical composition to a patient.

FIG. 3 is an exemplary method 300 of forming a mixture via a carpule system. At block 302 the method comprises providing a carpule comprising a carpule body defining a first chamber and a second chamber. The carpule can include at least one divider disposed between the first chamber and the second chamber for isolating a first material in the first chamber from a second material in the second chamber.

At block 304, a portion of the divider is displaced. In some embodiments, the divider is displaced via closing a lid on the carpule mixing device. In some embodiments, closing the lid triggers an increase in pressure on one side of the divider. The pressure flexes, displaces, or otherwise ruptures a portion of the divider.

At block 306, a mixture forms upon mixing the first material with the second material. Upon displacement of the divider, the first material and the second material interact within the sealed carpule to reconstitute the powdered substance, including but not limited to a powdered medicine or pharmaceutical such as BOTOX®.

At block 308, the carpule containing the mixture is inserted in a carpule syringe. The carpule is sealed on both ends as it is inserted into the carpule syringe. Methods of forming a mixture described in this Example offer many advantages compared to other methods. For example, powder can be reconstituted within a sealed carpule without using multiple syringes and/or introducing air into the mixture being formed.

FIG. 4 is an exemplary method 400 of administering a composition to a patient. At block 402, a carpule is provided. The carpule can comprise a carpule body defining a first chamber and a second chamber and a divider disposed between the first chamber and the second chamber as described in Section I and FIG. 1 above. The divider can isolate a first material in the first chamber from a second material in the second chamber. In an exemplary embodiment, the first material is a powder and the second liquid is a solvent of the powder.

At block 404, a portion of the divider is displaced to mix the first material and the second material to form the composition within the carpule body. In an exemplary embodiment, the carpule divider is displaced pneumatically or mechanically by a carpule mixing device.

At block 406, the carpule containing the composition is inserted into a carpule syringe. The carpule can be sealed at both ends as it is inserted within a barrel of the syringe.

At block 408, the composition is injected into the patient via the carpule syringe. In exemplary embodiments, the composition is a pharmaceutical composition used to treat a patient for a medical condition.

Methods of administering a composition described in this Example offer many advantages compared to other methods. For example, the reconstituted composition can be administered from a sealed carpule without using multiple syringes and/or introducing air into the mixture being formed.

Various embodiments of the present subject matter have been described in fulfillment of the various objectives of the invention. It should be recognized that these embodiments are merely illustrative of the principles of the present subject matter. Numerous modifications and adaptations thereof will be readily apparent to those skilled in the art without departing from the present subject matter.

What is claimed is:

1. A carpule system comprising:
a carpule comprising:
a carpule body defining a first chamber and a second chamber; and
a divider disposed between the first chamber and the second chamber, wherein the divider is configured to isolate a first material in the first chamber from a second material in the second chamber;
a carpule mixing device configured to receive the carpule and displace a portion of the divider for mixing the first material with the second material to form a mixture; and
a carpule syringe configured to receive the carpule and dispense an amount of the mixture, wherein the amount of the mixture is measurable via an audible sound and/or a tactile sensation provided by the carpule syringe.

2. The system of claim 1, wherein the mixture is disposed in a sealed mixing chamber having a volume that corresponds to volumes of the first and second chambers.

3. The system of claim 1, wherein the carpule mixing device is configured to pneumatically and/or mechanically rupture the divider and to agitate the carpule.

4. The system of claim 1, wherein the carpule comprises a first external seal for sealing the first chamber and a second external seal for sealing the second chamber from an outside environment.

5. The system of claim 4, wherein the first and second external seals are disposed on opposing sides of the divider for maintaining an airtight seal inside the carpule body.

6. The system of claim 1, wherein the first chamber and the second chamber are coaxially aligned.

7. The system of claim 1, wherein the first material is a liquid and the second material is a powder.

8. The system of claim 7, wherein the liquid is a solvent of the powder.

9. The system of claim 7, wherein the powder is a pharmaceutical composition.

10. The system of claim 7, wherein the powder comprises a botulinum toxin.

11. A method of forming and/or dispensing a mixture, the method comprising:
providing a carpule, the carpule comprising a carpule body defining a first chamber and a second chamber and a divider disposed between the first chamber and the second chamber, the divider being configured to isolate a first material in the first chamber from a second material in the second chamber;
displacing a portion of the divider;
forming a mixture by mixing the first material with the second material; and
inserting the carpule containing the mixture into a carpule syringe,
wherein the carpule is sealed on both a first end and a second end when the carpule is inserted into the carpule syringe.

12. The method of claim 11 further comprising dispensing an amount of the mixture from the carpule using the carpule syringe, wherein the amount being dispensed is measurable via an audible sound and/or a tactile sensation provided by the carpule syringe.

13. A method of administering a composition to a patient, the method comprising:
providing a carpule comprising a carpule body defining a first chamber and a second chamber and a divider disposed between the first chamber and the second chamber, wherein the divider is configured to isolate a first material in the first chamber from a second material in the second chamber;
displacing a portion of the divider to mix the first material and the second material to form the composition within the carpule body;
inserting the carpule containing the composition into a carpule syringe; and
injecting the composition into the patient via the carpule syringe.

14. The method of claim 13 further comprising storing the composition prior to injecting the composition into the patient.

15. The method of claim 13, further comprising agitating the carpule to homogenize the composition prior to inserting the carpule into the carpule syringe.

16. The method of claim 13, wherein displacing a portion of the divider comprises pneumatically and/or mechanically rupturing the divider.

17. The method of claim 13, wherein the first material comprises a liquid and the second material comprises a powder.

18. The method of claim 17, wherein the liquid is a solvent of the powder.

19. The method of claim 17, wherein the powder comprises a botulinum toxin.

20. The method of claim 13, further comprising measuring an amount of the composition injected into the patient via an audible sound and/or a tactile sensation provided by the carpule syringe.
